(19) United States
(12) Patent Application Publication
(10) Pub. No.: US 2014/0377845 A1
(43) Pub. Date: Dec. 25, 2014

(54) Isoprene Production

(71) Applicant: WiSys Technology Foundation, Inc., Madison, WI (US)

(72) Inventors: Eric L. Singsaas, Stevens Point, WI (US); Amy Wiberley, Madison, WI (US)

(21) Appl. No.: 14/471,692

(22) Filed: Aug. 28, 2014

Related U.S. Application Data
(63) Continuation of application No. 12/854,530, filed on Aug. 11, 2010.
(60) Provisional application No. 61/234,156, filed on Aug. 14, 2009.

(51) Int. Cl. C12P 5/00; C12N 15/75; C12N 15/82; C12N 15/74; C12N 15/81; C12N 15/70

(52) U.S. Cl. CPC C12P 5/007; C12N 15/815; C12N 15/70; C12N 15/8257; C12N 15/74; C12N 15/81; C12N 15/75
USPC 435/252.31; 435/254.23; 435/252.33; 435/257.2; 435/252.3; 435/254.21; 435/258.1

(57) ABSTRACT

A method of producing isoprene is disclosed. In one embodiment, the method comprises the steps of obtaining a host transgenic microorganism and observing the production of isoprene by the microorganism. In another embodiment, the invention is a transgenic host microorganism for producing isoprene.

### CROSS-REFERENCE TO RELATED APPLICATIONS

This application is a continuation of U.S. application Ser. No. 12/854,530 filed Aug. 11, 2010, which claims the benefit of U.S. Provisional application 61/234,156, filed Aug. 14, 2009. All the above applications are incorporated herein by reference in their entirety.

### STATEMENT REGARDING FEDERALLY SPONSORED RESEARCH OR DEVELOPMENT

This invention was made with United States government support awarded by the following agencies: Department of Defense W911NF-09-2-003, National Science Foundation IBN-0212204 and IOB-0640853. The United States government has certain rights in this invention.

### BACKGROUND OF THE INVENTION

The development of fuels from renewable agricultural sources is currently and will likely continue to be important in meeting future energy demands and reducing the production of greenhouse gas emissions from fossil carbon sources. Current “biofuels” under development include “biodiesel” derived via fatty acid synthesis from vegetable oil and ethanol fermented from sucrose obtained from plants such as corn and sugarcane.

More diverse and advanced biofuels and bio-products may be developed by exploiting metabolic pathways other than fatty acid synthesis and fermentation. Our invention, described below, exploits the 2-C-methyl-D-erythritol 4-phosphate (MEP) pathway illustrated in FIG. 1. Plants use this pathway to synthesize isoprene, and both plants and bacteria use it to synthesize other terpenoids. In plants, the MEP pathway is highly productive relative to other secondary metabolic pathways. The pathway converts between 2% and 20% of CO₂ assimilated by photosynthesis into isoprene (2-methyl 1,3-butadiene). Globally, plants emit more than 500 teragrams of isoprene into the atmosphere per year. This amount exceeds anthropogenic hydrocarbon emissions.

Regulation of flux through the MEP pathway is an active area of research, and several of the pathway's enzymes have been suggested to have key roles in regulation of this flux. Overexpression of 1-deoxy-D-xylulose-5-phosphate synthase (DXS) has been found to increase production of terpenoids synthesized from MEP pathway products in a number of plant species (Estevez et al., J. Biological Chem. 276:22904-22909 (2001); Lois et al., Plant J. 22:503-513 (2001); Rodrigues-Concepción et al., Planta 217:476-482 (2003); Enfissi et al., Plant Biotechnology J. 3:17-27 (2005); Morris et al., J. Experimental Botony 57:3007-3018 (2006); Muñoz-Bertomeu et al., Plant Physiology 142:890-900 (2006)). Increased levels of DXS transcript correlated with an increase in MEP pathway-produced terpenoid accumulation in other plant species (Walter et al., Plant J. 21:571-578 (2000); Walter et al., Plant J. 31:243-254 (2002); Gong et al., Planta Medica 72:329-335 (2006); Kishimoto & Ohmiya, Physiologia Plantarum 128:436-447 (2006)).

Similar overexpression experiments and transcript analyses suggest a regulatory role for 1-deoxy-D-xylulose-5-phosphate reductoisomerase (DXR) as well (Veau et al., Biochimica et Biophysica Acta: Gene Structure and Expression 1517:159-163 (2000); Walter et al., Plant J. 21:571-578 (2000); Mahmoud & Croteau, Proc. Nat'l Acadamy Sci. U.S.A. 98:8915-8920 (2001); Bede et al., Plant Mol. Biol. 60:519-531 (2006); Carretero-Paulet et al., Plant Mol. Biol. 62:683-695 (2006)). Hydroxymethylbutenyl diphosphate reductase (HDR) overexpression also increases accumulation of terpenoids produced by the MEP pathway, and its transcript levels correlate with terpenoid accumulation (Botella-Pavia. et al., Plant J. 40:188-199 (2004)). Increased isopentenyl diphosphate isomerase (IDI) expression is also likely to increase carbon flux to isoprene, since isoprene synthase (IspS) uses dimethylallyl diphosphate (DMADP) exclusively (Sanadze, Current Research in Photosynthesis IV:231-237 (1990)). However, HDR produces isopentenyl diphosphate (IDP) and DMADP in approximately a 6:1 ratio ((Rohdich et al., Proc. Nat'l Acad. Sci. U.S.A. 99:1158-1163 (2002); Rohdich et al., Proc. Nat'l Acad. Sci. U.S.A. 100:1586-1591 (2003); Adam et al., Proc. Nat'l Acad. Sci. U.S.A. 99:12108-12113 (2002)), and IDI is needed to convert IDP to DMADP (Rodrigues-Concepcion et al., Planta 217:476-482 (2003); Page et al., Plant Physiology 134:1401-1413 (2004)). IDI activity has been found to correlate with isoprene production by oak trees (Brüggemann & Schnitzler, Tree Physiology 22:1011-1018 (2002)).

However, an increase in gene expression does not necessarily correlate with increased production of terpenoids. In one study, hydroxymethylbutenyl diphosphate synthase (HDS) overexpression has been shown to have no effect on terpenoid production (Flopres-Pérez et al., Biochem Biophys Res Comm 371:510-514 (2008)). It is possible that increasing an enzyme's expression might use more of the cell's resources. Additionally, increasing the expression of a gene does not necessarily increase the amount of an enzyme or its activity.

Recently, there has been an increased demand for the production of biofuels. Accordingly, there is a need for improved methods of isoprene production, which may later be converted into biofuels.

### SUMMARY OF THE INVENTION

The present invention is a method of isoprene production. It relies on the inventors' observations that isoprene may be produced by exploiting the MEP metabolic pathway.

In a first aspect, the present invention is a method of isoprene production including the step of obtaining a host transgenic microorganism, wherein the transgenic microorganism comprises transgenes encoding isopentenyl diphosphate isomerase (IDI), isoprene synthase (IspS), and 1-deoxy-D-xylulose-5-phosphate synthase (DXS). The method also includes the step of observing the production of isoprene by the microorganism, wherein isoprene production is at the rate of at least 3 μg/L/hr, preferably at least 35 μg/L/hr, more preferably at least 70 μg/L/hr.

In a different embodiment of the first aspect, the host transgenic microorganism further includes a transgene encoding hydroxymethylbutenyl diphosphate reductase (HDR) and wherein isoprene production is at the rate of at least 70 μg/L/hr, preferably 140 μg/L/hr.

In another embodiment of the first aspect, the host transgenic microorganism of the first step further comprises a transgene encoding 1-deoxy-D-xylulose-5-phosphate reductoisomerase (DXR).

In another embodiment of the first aspect, the host transgenic microorganism of the first step further comprises at least one transgene selected from the group consisting of transgenes encoding hydroxymethylbutenyl diphosphate reductase (HDR), 1-deoxy-D-xylulose-5-phosphate reductoisomerase (DXR), 4-diphosphocytidyl-2-C-methyl-D-erythritol synthase (CMS), 4-diphosphocytidyl-2-C-methyl-D-erythritol kinase (CMK), and 2-C-methyl-D-erythritol 2,4-cyclodiphosphate synthase (MCS).

In another embodiment of the first aspect, at least one of the transgenes is isolated from Populus trichocarpa.

In another embodiment of the first aspect, one of the trangenes is Populus trichocarpa IDI.

In another embodiment of the first aspect, one of the transgenes is Populus trichocarpa IspS.

In another embodiment of the first aspect, at least one of the transgenes is isolated from a non-E. coli source and the transgene has been codon amplified for insertion into an E. coli host transgenic microorganism.

In another embodiment of the first aspect, the host transgenic microorganism is E. coli.

In another embodiment of the first aspect, the host transgenic microorganism is a photosynthetic cyanobacterium.

In another embodiment of the first aspect, the host transgenic microorganism of the first step additionally comprises flavodoxin and flavodoxin reductase.

In another embodiment of the first aspect, the method further includes the step of providing a fermentation medium.

In another embodiment of the first aspect, the fermentation medium comprises glucose.

In another embodiment of the first aspect, the fermentation medium comprises paper mill sludge hydrolysate produced by enzyme or acid-catalyzed hydrolysis of waste fibers from a pulp mill.

In another embodiment of the first aspect, the method further includes the step of recovering the isoprene of the second step.

In another embodiment of the first aspect, the method further includes the step of chemically modifying the recovered isoprene into the group selected from dimer (10-carbon) hydrocarbons, trimer (15-carbon) hydrocarbons, and mixtures of dimer and trimer hydrocarbons.

In another embodiment of the first aspect, the dimer and/or trimer hydrocarbons are hydrogenated to long-chain, branched alkanes suitable for use in fuel or solvents.

In another embodiment of the first aspect, the dimer hydrocarbons are used in organosolv pulping.

In another embodiment of the first aspect, the isoprene is used to produce rubber.

In another embodiment of the first aspect, the isoprene is polymerized with catalyst systems to form homopolymers of cis-3-polyisoprene.

In another embodiment of the first aspect, the isoprene is co-polymerized with styrene or butadiene to produce an elastiomer.

In another embodiment of the first aspect, the isoprene is polymerized with an oxidant to form hydroxyl-terminated polyisoprene.

In another embodiment of the first aspect, the oxidant is hydrogen peroxide.

In another embodiment of the first aspect, the hydroxyl-terminated polyisoprene is used as a pressure-sensitive adhesive.

In another embodiment of the first aspect, the isoprene is polymerized into liquid fuels that are infrastructure compatible with current gasoline, diesel or jet engines.

In a second aspect, the present invention is a method of isoprene production including the step obtaining a host transgenic microorganism, wherein the transgenic microorganism consists of transgenes encoding isopentenyl diphosphate isomerase (IDI), isoprene synthase (IspS), and 1-deoxy-D-xylulose-5-phosphate synthase (DXS). The method also includes the step of observing the production of isoprene by the microorganism, wherein isoprene production is at the rate of at least 3 μg/L/hr, preferably at least 35 μg/L/hr, more preferably at least 70 μg/L/hr.

In another embodiment of the second aspect, the host transgenic microorganism further consists of a transgene encoding hydroxymethylbutenyl diphosphate reductase (HDR) and isoprene production is at the rate of at least 70 μg/L/hr.

In another embodiment of the second aspect, the host transgenic microorganism further consists of a transgene encoding 1-deoxy-D-xylulose-5-phosphate reductoisomerase (DXR).

In a third aspect, the present invention is a transgenic host microorganism. The transgenic host microorganism includes transgenes encoding isopentenyl diphosphate isomerase (IDI), isoprene synthase (IspS), and 1-deoxy-D-xylulose-5-phosphate synthase (DXS).

In another embodiment of the third aspect, the transgenic host microorganism further includes a transgene encoding hydroxymethylbutenyl diphosphate reductase (HDR).

In another embodiment of the third aspect, the transgenic host microorganism further includes a transgene encoding 1-deoxy-D-xylulose-5-phosphate reductoisomerase (DXR).

In another embodiment of the third aspect, the transgenic host microorganism further includes at least one transgene selected from the group consisting of transgenes encoding hydroxymethylbutenyl diphosphate reductase (HDR), 1-deoxy-D-xylulose-5-phosphate reductoisomerase (DXR), 4-diphosphocytidyl-2-C-methyl-D-erythritol synthase (CMS), 4-diphosphocytidyl-2-C-methyl-D-erythritol kinase (CMK), and 2-C-methyl-D-erythritol 2,4-cyclodiphosphate synthase (MCS).

In another embodiment of the third aspect, the transgenic host microorganism additionally includes flavodoxin and flavodoxin reductase.

In another embodiment of the third aspect, the transgenic host microorganism is an E. coli.

In another embodiment of the third aspect, the transgenic host microorganism is a photosynthetic cyanobacterium.

In another embodiment of the third aspect, at least one of the transgenes is isolated from Populus trichocarpa.

In another embodiment of the third aspect, the transgene is Populus trichocarpa IDI.

In another embodiment of the third aspect, the transgene is Populus trichocarpa IspS.

Other objects, advantages and features of the present invention will become apparent after review of the specification, claims and drawings.

### BRIEF DESCRIPTION OF THE DRAWINGS

FIG. 1. The methylerythritol 4-phosphate (MEP) pathway for biosynthesis of isoprene is a metabolic pathway leading to the synthesis of isopentenyl diphosphate (IDP) and dimethylallyl diphosphate (DMADP).

FIG. 2A. Nucleotide sequence for DXS.

FIG. 2B. Nucleotide sequence for DXR.

FIG. 2C. Nucleotide sequence for CMS.

FIG. 2D. Nucleotide sequence for CMK.

FIG. 2E. Nucleotide sequence for MCS.

FIG. 2F. Nucleotide sequence for HDS.

FIG. 2G. Nucleotide sequence for HDR.

FIG. 2H. Nucleotide sequence for IDI.

FIG. 3. Isoprene emission from BL21(DE3) lines with or without Populus trichocarpa isoprene synthase (PIspS) or Populus trichocarpa isopentenyl diphosphate isomerase (PIDI) and PIspS.

FIG. 4. Isoprene emission from ScarabXpress™ lines with different combinations of MEP pathway genes and PIspS.

FIG. 5. Isoprene production by cell lines overexpressing DXS, IDI, and/or PIspS, measured in sealed vials.

FIG. 6. mRNA accumulation for cell lines overexpressing or not overexpressing MEP pathway genes and PIspS. RPOB is the control gene, RNA polymerase B.

FIG. 7. Isoprene production by cell lines overexpressing different combinations of MEP pathway genes with PIspS measured in sealed vials.

FIG. 8. Average isoprene production by all cell lines overexpressing or not overexpressing HDS measured in sealed vials.

FIG. 9. Isoprene production in a 1.3 L fermenter culture with strain O1E89.

FIG. 10 Isoprene production from hydrolyzed pulp mill sludge in a 1.3 L fermenter culture with strain A17E89.

FIG. 11. Synthetic operon for testing different combinations of genes and individually controlling genes using different promoters.

FIG. 12. Sequence for synthetic operon for testing different combinations of genes and individually controlling genes using different promoters.

### DETAILED DESCRIPTION OF THE INVENTION

### A. In General

Applicants herein disclose a method for producing isoprene using transgenic microorganisms comprising transgenes that encodes certain enzymes in the MEP metabolic pathway. Applicants discovered that unique combinations of these enzymes result in increased isoprene production.

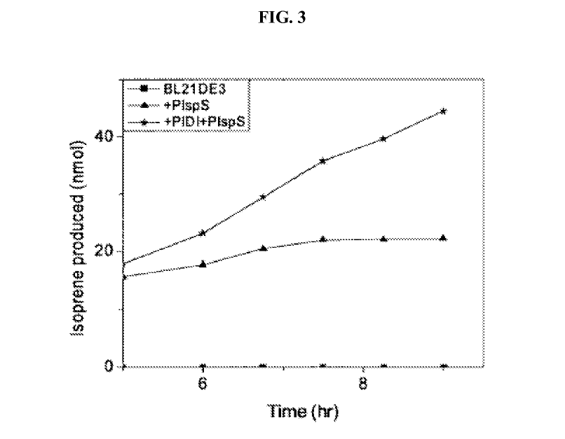

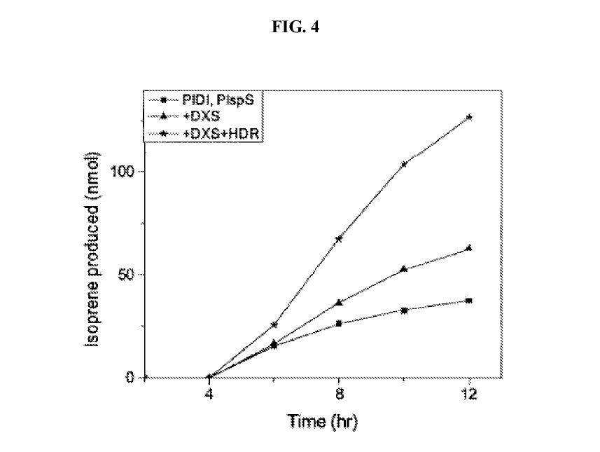

The examples below show that reduction by microbes of hemiterpenes such as isoprene and methyl butenol is of interest for synthesis of biofuels and fine chemicals. Genes involved in the production of isoprene by the tree Populus trichocarpa were cloned and transformed into E. coli. E. coli genes with silent mutations to remove unwanted restriction sites were also cloned and transformed. Introduction of IspS, IDI, and DXS greatly increased isoprene production compared to that of non-transgenic cells (see FIGS. 3 and 4). Adding other MEP pathway genes, especially HDR and DXR, further increased isoprene yield.

Throughout this application, experimental strains are referred to by the numbering of the MEP pathway genes overexpressed in that line, e.g. O1E89. The nomenclature is as follows: 1-DXS; 2-DXR; 3-CMS; 4-CMK; 5-MCS; 6-HDS; 7-HDR;, 8-IDI; and 9-IspS. The letters “O”, “E”, “A”, “D”, refer to the four Duet vectors: O-pCOLADuet™; E-pETDuet™; A-pACYCDuet™; and D-pCDFDuet™. Thus, O1E89 contains pCOLADuet with DXS and vector pETDuet™ with IDI and IspS. Strain A17E89 contains vector pACYCDuet™ with DXS and HDR and vector vector pETDuet™ with IDI and IspS.

### B. Genes

By “transgenic” or “transgenes” we mean a gene that has been recombinantly introduced into a microorganism. In a preferred embodiment, the transgenes IDI, DXS, and IspS are recombinantly introduced into a microorganism. Preferably, the transgene IDI is E. coli IDI and the transgene IspS is P. trichocarpa IspS (PIspS). Populus trichocarpa IDI (PIDI) may also be used, as it has higher activity than E. coli IDI, and PIDI is easily expressed at high levels in E. coli. However, in an alternative embodiment of the invention, one may substitute E. coli IDI. There is no known or putative bacterial IspS.

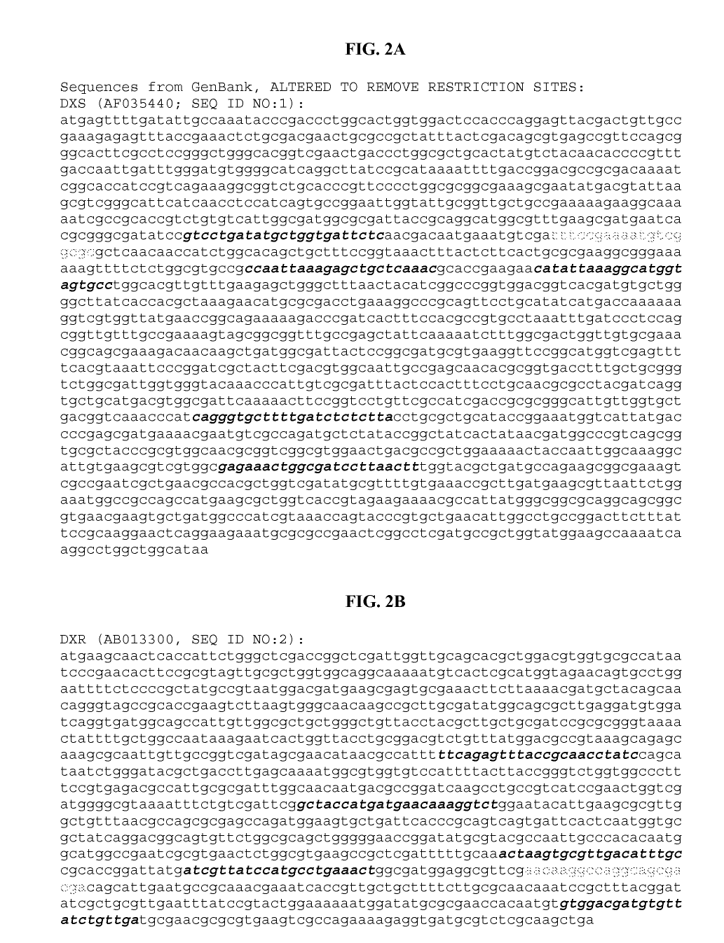

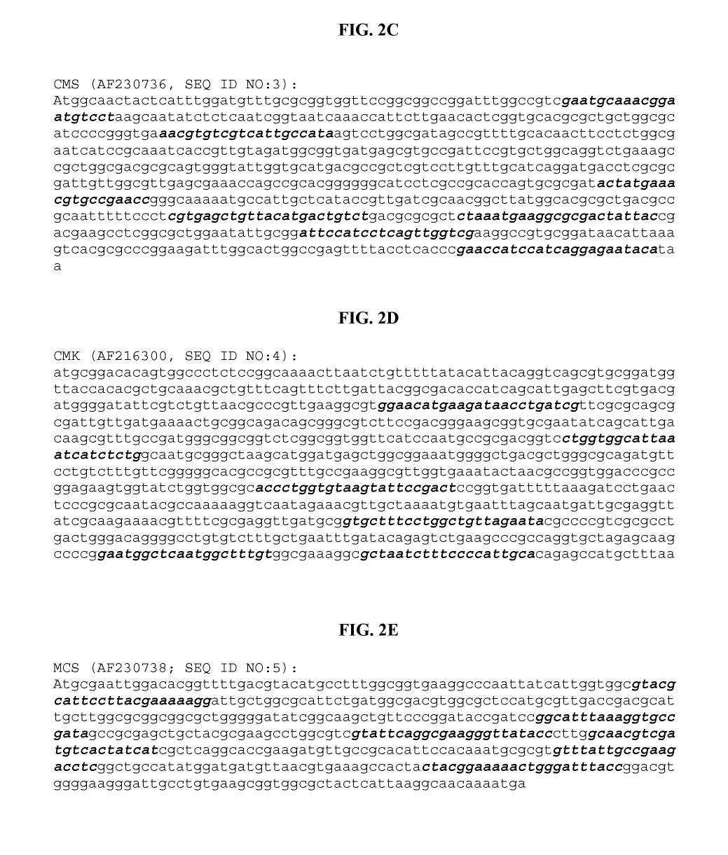

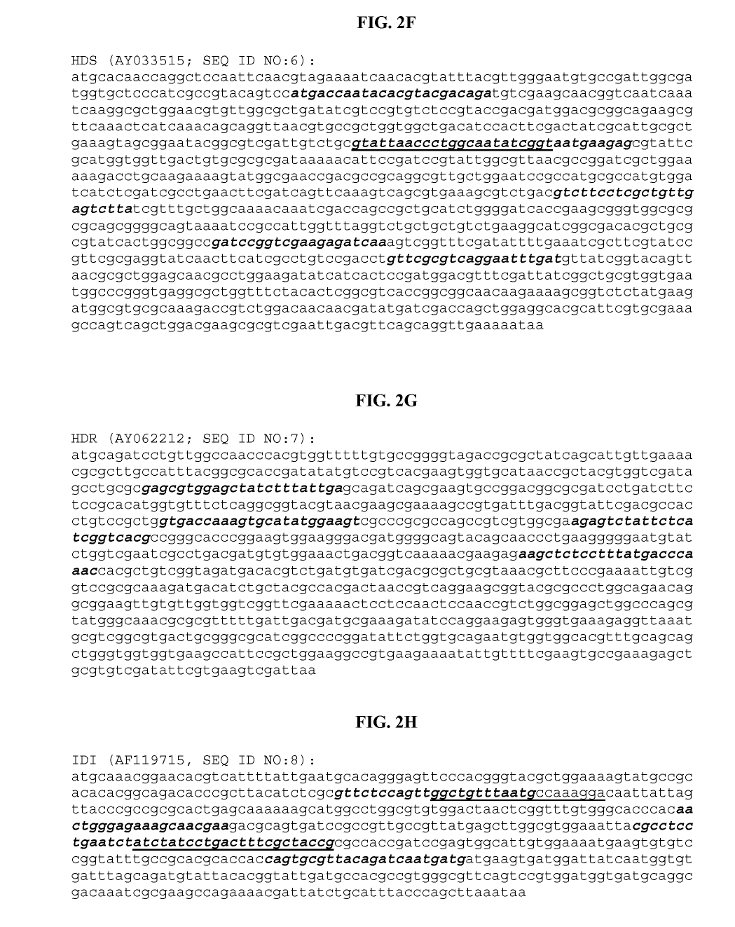

Preferred E. coli MEP pathway gene sequences, which have been altered to remove unwanted restriction sites, are shown in FIGS. 2A-2H. The PIDI and PIspS sequences can be accessed via GenBank. The accession number for PIDI is EU693026, which is hereby incorporated by reference. The accession number for PIspS is EU693027, which is hereby incorporated by reference. The accession number for DXS is AF035440, which is hereby incorporated by reference. The accession number for DXR is AB013300, which is hereby incorporated by reference. The accession number for CMS is AF230736, which is hereby incorporated by reference. The accession number for CMK is AF216300, which is hereby incorporated by reference. The accession number for MCS is AF230738, which is hereby incorporated by reference. The accession number for HDS is AY033515, which is hereby incorporated by reference. The accession number for HDR is AY062212, which is hereby incorporated by reference. The accession number for IDI is AF119715, which is hereby incorporated by reference. E. coli DXS and HDR genes are preferably amplified from E. coli sequences commercially synthesized and designed to have silent mutations that remove undesired restriction enzyme recognition sites.

In another embodiment of the invention, one may wish to optimize gene expression by modifying the transgenes with codons optimally used by the host. Most amino acids are encoded by more than one codon. Each organism carries a bias in the usage of the 61 available amino acid codons. In certain embodiments, one may wish to modify a gene, for example PIDI, with E. coli-specific codons. By E. coli-specific codons we mean codons toward which an organism is biased in the usage of the 61 available amino acid codons (Novy et al., InNovations 12 (June 2001)). The Novy study showed that certain codons are used much more than others, and the presence of clusters or and/or numerous rare E. coli codons can significantly reduce gene expression.

The PIDI and PIspS transgenes are preferably created according to the following protocol: Total RNA is extracted as described by Haruta et al. (Plant Mol. Biol. 46:347-359 (2001)) and quantitated using a Beckman DU 640 spectrophotometer (Beckman Coulter, Inc., Fullerton, Calif., USA). All chemicals can be obtained from Sigma-Aldrich (St. Louis, Mo., USA) unless otherwise noted. RNA is reverse transcribed and amplified by polymerase chain reaction (PCR) using primers that clone the full length cDNAs, less the putative transit peptides. Suitable restriction enzyme recognition sites are added to the ends of the genes.

In a preferable embodiment, after transforming the host cell with genes encoding IspS, IDI, and DXS, the host cell may also be transformed with 1-deoxy-D-xylulose 5-phosphate reductoisomerase (DXR) and/or hydroxymethylbutenyl diphosphate reductase (HDR). In a preferred embodiment the only genes encoding MEP pathway members with which the host cell is transformed are genes encoding IspS, IDI, DXS, and DXR. In another preferred embodiment the only genes encoding MEP pathway members with which the host cell is transformed are genes encoding IspS, IDI, DXS, and HDR. In other embodiments, Applicants expect that one may wish to transform the host cell with genes encoding other MEP pathway members, such as 4-diphosphocytidyl-2-C-methyl-derythritol synthase (CMS), 4-diphosphocytidyl-2-C-methyl-D-erythritol kinase (CMK), and 2-C-methyl-D-erythritol 2,4-cyclodiphosphate synthase (MCS), flavodoxin, and/or flavodoxin reductase. Preferably, the transgenes, for example CMK and MCS, are cloned from a restriction site-free construct. The sequence listing for each of these transgenes is shown in FIGS. 2A-2H. Flavodoxin and flavodoxin reductase are preferably cloned from E. coli genomic DNA.

One may wish to use transgenic genes from other sources, such as kudzu and other legume plant sequences, eucalyptus sequences, and sequences from Melaleuca species. In addition, similar isoprene synthase sequences with different gene structure from gymnosperms such as Picea could be used. One could also use sequences genetically engineered to increase yield and utility when the sequences are based on isoprene synthase sequences based on any of these species of isoprene synthases from ferns or mosses.

### C. Hosts

In a preferred embodiment the host transgenic microorganism is E. coli, preferably a BL21(DE3) cell line of E. coli, more preferably a ScarabXpress™ T7lac cell line of E. coli (Scarab Genomics L.L.C., Madison, Wis.). Applicants' first isoprene-overproducing E. coli strain was a BL21(DE3) cell line that was transformed with pET28a-PIspS. Suitable transgenic hosts are Bacillus spp, and Lactobacillus spp., Geobacillus sterothermophilus strain G1.13, Mycobacterium smegmatis, Clostridia spp.

In another embodiment the host transgenic microorganism is a photosynthetic cyanobacterium. The optimal combination of overexpressed MEP pathway genes found in E. coli can also be applied to produce isoprene from other organisms such as cyanobacteria. Cyanobacteria such as Synechococcus have homologues for all the MEP pathway genes, so the same supplementation of expression of key genes to increase isoprene production can likely be used. The E. coli MEP pathway genes and PIspS can be introduced to cyanobacterial cells using homologous recombination (Clerico et al., Methods in Mol. Biol. 362:155-171 (2007)) or plasmids that can replicate in certain cyanobacterial strains (Takeshima et al., DNA Research 1:181-189 (1994)). Other cyanobacteria such as Synechocystis PCC6803, Thermosynechococcus elongatus BP-1, Synechococcus sp. PCC 7002, Anabaena variablis ATCC29413, Anabaena PCC7120, and Nostoc punctiforme ATCC7312 are also suitable for use as the host transgenic microorganism.

In a different embodiment the host transgenic microorganism is a yeast. The MEP pathway would be expressed in yeast such as Saccharomyces spp. or Pichia spp., with the addition of IspS. Yeast does not have the MEP pathway, so one would need to add all of the genes in the pathway or rely on the mevalonic acid pathway to produce the precursors. Different promoters could be used to yield greater expression of the combination of genes shown, by work in E. coli, to have the greatest effect on metabolite flux through the pathway. Expression of foreign genes in yeast is a well known technology and can be carried out with established procedures.

In different embodiments, the host transgenic organism is a photosynthetic green alga such as Chlamydomonas spp. or Anabaena spp.

In a different embodiment, the host transgenic organism is a thermoacidophilic bacterium, such as Spirochaeta americana, Deinococcus-Thermus, Thermus thermophilus, Deinococcus radiodurans, Thermus aquaticus, Chloroflexus aurantiacus, or Pyrococcus furiosus. These hosts possess a native MEP pathway and their tolerance of high temperature and low pH and metabolism of sulfur make them suitable for production of isoprene from biomass such as pulp mill sludge, which contains large amounts of sulfur and has been pretreated with strong acid and heat.

In a different embodiment, the host transgenic organism is a thermoacidophilic archaebacterium, such as Pyrococcus furiosus or Pyrolobus fumarii. These hosts produce isoprenoid memebrane lipids and thus are capable of producing large quantities of DMADP. Their tolerance of high temperature and low pH make them suitable for production of isoprene from biomass that has been pretreated with strong acid and heat.

It is further envisioned that one could use modified E. coli as the host organism in the present invention. Modified E. coli include those having a genome genetically engineered to be smaller than the genome of its natural parent strain and E. coli engineered to have a “clean genome”, i.e., lacking, for example, genetic material such as certain genes unnecessary for growth and metabolism of the bacteria. Using modified E. coli would allow the expression of isoprene to be optimized. In a preferred embodiment, E. coli modified to increase protein expression would be used.

### D. Vectors and Constructs

In one embodiment, constructs for isoprene production are created using cell lines transformed with a transgene vector. In a preferred embodiment the cell line is E. coli BL21(DE3). The cell line is transformed with pET28a-PIspS. A preferred construct includes the PIspS cDNA, less its predicted transit peptide, ligated into the pET28a expression vector (Novagen) with an isopropyl β-D-lthiogalactopyranoside (IPTG)-inducible promoter (Calfpietra et al., Plant, Cell & Environment 30:654-661 (2007)).

For overexpression of multiple MEP pathway genes, the Duet™ vector series from Novagen is used in one embodiment. These vectors allow simultaneous expression of two genes or transgenes from one plasmid; each gene has a separate IPTG-inducible promoter. There are five vectors in this series. Each has a different origin of replication, and there are four antibiotic resistance genes among the vectors. This means that four Duet™ vectors can coexist in a single cell, so eight genes can be overexpressed simultaneously.

Duet™ vector constructs can be transformed into ScarabXpress™ T7lac E. coli cells (Scarab Genomics, Madison, Wis.). These cells often have improved heterologous protein expression compared to BL21(DE3) cells.

While the Duet vector system allowed simultaneous overexpression of all the genes in the MEP pathway as well as PIspS, it is most likely not the ideal host for isoprene production from E. coli. Maintenance of up to four plasmids for gene expression places a significant metabolic burden on the host, and the use of one fairly strong promoter for expression of all the genes does not allow for fine-tuning of expression based on the particular requirements for any enzyme(s).

The inventors designed and constructed a synthetic operon (genes linked together in a continuous piece of DNA) further described in Example 5. By PCR amplification with appropriate primers, or by restriction digestion out of a vector, the operon can be cloned into virtually any expression vector or BAC for propagation and expression in any of a number of microbial hosts. Suitable BAC vectors include, but are not limited to, pBAC108L, pBeloBAC11, pBACe3.6, and pSMART VC Vectors.

For example, the operon may be inserted into: E. coli or another prokaryote organism using a commercial Bacterial Artificial Chromosome; a prokaryotic organism using a linear cloning vector, such as pJazz from Lucigen Corporation (Madison, Wis., USA); and the genome of a cyanobacterium such as Anabaena, Synecococcus, or Synechocystis using a shuttle vector system.

### E. Production and Collection

The present invention provides a method of producing isoprene from a microorganism at a rate of at least 70 m/L/hr, preferably at least 140 μg/L/hr. After eight hours of growth, a 6.6 mL culture of the microorganism will have produced at least 20-40 nmols of isoprene, more preferably 40-65 nmols of isoprene.

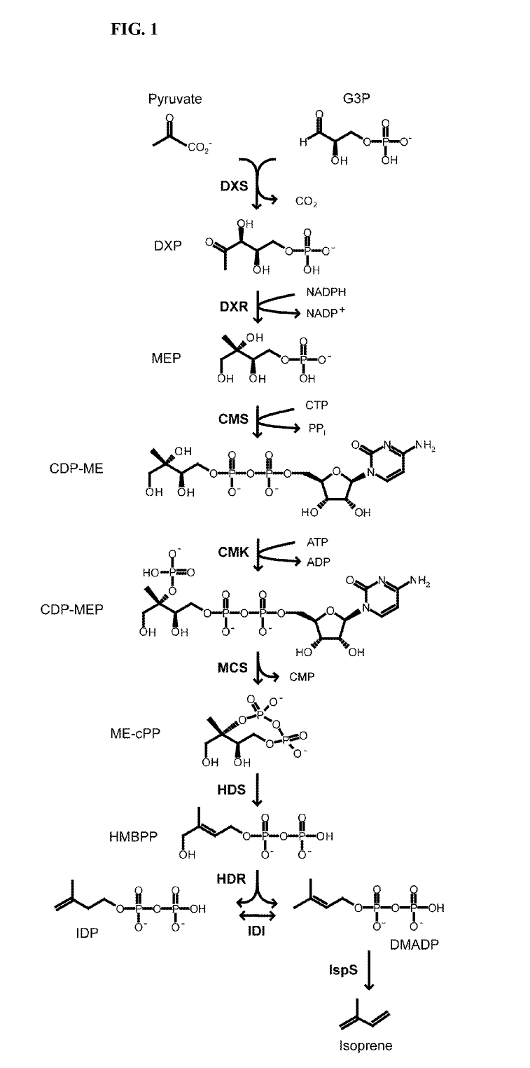

Isoprene can be produced in a bioreactor from bacteria, yeast from sugars obtained from corn or cellulosic biomass. These organisms metabolize glucose into pyruvate and glyceraldehyde-3-phosphate (G3P; FIG. 1) via the glycolytic pathway. Paper mill pulp or sludge can also serve as the substrate for the bioreaction.

Briefly, pulp mill primary sludge is optionally washed with water, and then treated with commercial cellulase enzymes, according to the manufacturer's directions. Enzymatic digestion is conducted with 5-10% (w/v) sludge solids in 0.5 M Citrate buffer, pH 4.8 with enzyme dosages of 0.25-1.5% (v/v). This slurry is incubated at 50° C. for 48-72 hours with continuous stirring or shaking. The solids are removed by centrifugation and sterilized with either a 0.2-micron filter or by autoclaving (121° C. for 20 min.). (Note that different manufacturers have slightly different enzyme dosages and reaction conditions, so these are dependent on the product.) A K12 or M9 salt mixture and appropriate antibiotics and micronutrients is added to the sterile sugar mixture and put into a sterile fermentation vessel. Adding a starter culture of the isoprene-producing organism starts the isoprene production.

Isoprene can also be produced by photosynthetic means in green algae or cyanobacteria by taking pyruvate and G3P from the Calvin cycle. This method of producing isoprene is more efficient than producing it via the mevalonate pathway, which necessitates making sugars, breaking the sugars down completely into acetyl groups, and then reforming the acetyl groups into mevalonate.

In a preferred embodiment, paper mill pulp or sludge is the substrate. The primary sludge from a pulp mill is a waste product containing spent pulping chemicals, impurities, sugar oligomers and short cellulose fibers that pass through the pulping process. Our analysis indicates that primary sludge contains up to 48% by weight fermentable carbohydrates. Sludge is a waste product from a mill, which must be collected and shipped to a landfill at a cost of more than $100 per ton to the mill. Pulp mills in Central Wisconsin produce between 50-100 tons of sludge per day. Converting sludge into a bioproduct such as isoprene reduces landfill waste, saves transport and landfill fees and produces a valuable product that adds revenue to the mill.

One may wish to recover the isoprene produced by the present invention. Isoprene can be captured from a nitrogen or air gas stream, which is bubbled through a culture of isoprene-producing cells of the present invention. This may be done by means of a fermentation system with a built in gas sparger. Isoprene can be collected by distillation, adsorption onto a polymer membrane or by filtration in the manner of a filter gas purifier. These methods may be used individually or in combination to obtain high purity liquid isoprene.

### F. Applications

Isoprene is a valuable material suitable for use as a chemical feedstock to replace or supplement artificial isoprene in the fine chemicals market. Natural rubber is a polymer of isoprene, and artificial rubbers are made from co-polymers of isoprene, butadiene, and other unsaturated hydrocarbons. Currently, isoprene's industrial use is constrained by its tight supply. For this reason, most synthetic rubbers are made from butadiene, a more readily available monomer, but one that is substantially more toxic than isoprene (De Meester et al., In: Industrial and Environmental Xenobiotics, 195-203 (1981)).

Isoprene produced by the method of the present invention may also be polymerized immediately upon collection with catalyst systems to form homopolymers of cis-3-polyisoprene. It may also be co-polymerized with styrene or butadiene for elastiomer production. In a similar manner, isoprene can be polymerized with an oxidant such as hydrogen peroxide to form hydroxyl-terminated polyisoprene for use as a pressure-sensitive adhesive. Alternate forms of hydroxyl-terminated polyisoprene might be used as a hybrid rocket fuel in conjunction with an oxidizer such as nitrous oxide.

The polymer cis-polyisoprene is valuable for making sporting goods, medical supplies, footwear, racing tires and a variety of products containing elastic substances (Mark et al., In: Encyclopedia of Polymer Science and Technology V7:782-854 (1967)).

Biologically-produced isoprene of the present invention can also be polymerized into liquid fuels that would be infrastructure-compatible with current gasoline, diesel or jet engines. Isoprene can be reacted with imidazolium salts and phosphenes over a catalyst such as palladium-carbene in the presence of methanol, ethanol, butanol, isopropanol or methyl butenol to form telomerisation products (Clement et al., Chem.—A European J. 14:7408-7402 (2008)). These reactions result in a mixture of ten-carbon branched alkenes or 11-13-carbon esters with chemical characteristics that make them suitable for fuels. Isoprene may also be telomerized with glycerol, a byproduct of biodiesel production (Jackstell et al., J. Organometallic Chem. 692:4737-4744 (2007)), to produce terpene derivatives for fuel use.

Recovered isoprene of the present invention can be chemically modified into dimer (10-carbon) and trimer (15-carbon) hydrocarbons using catalysts to make unsaturated alkenes. (Clement et al., Chem. Eur. J. 14:7408-7420 (2008); Gordillo et al., Adv. Synth. Catal. 351:325-330 (2009)). These hydrocarbons can be hydrogenated to make long-chain, branched alkanes suitable for fuel or solvent use.

The isoprene dimers and telomerisation products may also be used in organosolv pulping, for example, employing the process described in U.S. Patent Application No. US2009/0145021, which is hereby incorporated by reference. The organosolv pulper receives an organic solvent such as ethanol, methylbutanol, butanol, or dienes including 2,6-dimethyl-2,6-octadiene; 2,7-dimethyl-2,6-octadiene; 2,3,5-trimethyl-1,5-heptadiene; and 2,3,6-trimethyl-1,5-heptadiene, to extract lignin from the cellulose of the lignocellulosic material producing a “black liquor” comprised of aqueous solvent and lignin. In addition to the solvent, the organosolv pulper may receive acid or base up to 1% based on oven-dry biomass weight.

### EXAMPLES

### Example 1

β Isoprene production was measured using a BL21(DE3) cell line of E. coli. Untransformed BL21(DE3) does not emit isoprene (as indicated by the squares in FIG. 3). An E. coli BL21(DE3) cell line was transformed with pET28a-PIspS. This construct includes the Populus trichocarpa IspS (PIspS) cDNA, less its predicted transit peptide, ligated into the pET28a expression vector (Novagen) with an isopropyl β-D-1 thiogalactopyranoside (IPTG)-inducible promoter (as indicated by the triangles in FIG. 3) (Calfapietra et al., Plant, Cell & Environment 30:654-661 (2007)).

Populus trichocarpa isopentenyl diphosphate isomerase (PIDI), which produces much of the substrate for IspS, was introduced to cells already containing the PIspS gene (as indicated by the stars in FIG. 3). This resulted in a further doubling of isoprene emission, with a linear increase in emission over time. The PIDI gene product is an enzyme that interconverts isopentenyl diphosphate isomerase (IDI) and dimethylallyl diphosphate (DMADP), and can increase the production of any compound that preferentially uses DMADP over isopentenyl diphosphate (IDP), including isoprene and methyl butenol. This line emitted more isoprene than nontransgenic E. coli (see FIG. 3).

The slope of each line indicates the rate of isoprene production. After eight hours, a 5-mL culture of the PIspS-transformed cell line had produced approximately 20 nmol of isoprene and the PIDI and PIspS transformed cell line had produced approximately 40 nmol. The production rate was 29 μg/L/hr for the PIspS transformed cell line and about 95 μg/L/hr for the PIDI and PIspS transformed cell line.

### Example 2

E. coli DXS and HDR genes were cloned into the pACYDuet vector. The genes were amplified from E. coli sequences synthesized by BioBasic (Markham, Ontario, Canada) and designed to have silent mutations that removed undesired restriction enzyme recognition sites. Isopropyl β-D-1-thiogalactopyranoside (IPTG) was added at hour 4 to induce gene expression. Duet™ vectors were transformed into ScarabXpress™ T7lac E. coli cells.

The cells were transformed using pETDuet-PIDI-PIspS (FIG. 4, squares); pETDuet-PIDI-PIspS and pACYCDuet-DXS (FIG. 4, triangles); and pETDuet-PIDI-PIspS and pACYCDuet-DXS-HDR (FIG. 4, stars). First, PIDI and PIspS genes were cloned into the pETDuet vector. These genes were cloned from reverse-transcribed P. trichocarpa mRNA extracts. The PIDI was used rather than the E. coli IDI because E. coli IDI has fairly low activity and PIDI is easily expressed at high levels in E. coli (Wiberley, unpublished). Introduction of pRSETA-PIDI to pET28a-PIspS-containing BL21(DE3) strains doubled isoprene production (FIG. 3). When these transgenes were put in pETDuet and in ScarabXpress™ cells with pACYC-DXS-HDR, isoprene production increased even further (FIG. 4).

After eight hours, a 6.6-mL culture of the PIDI and PIspS-transformed cell line produced approximately 40 nmol of isoprene; a 6.6-mL culture of the PIDI, PIspS, and DXS transformed cell line produced approximately 42 nmol of isoprene; and a 6.6-mL culture of the PIDI, PIspS, DXS, and HDR transformed cell line produced approximately 65 nmol of isoprene. The production rate was about 48 μg/L/hr for the PIDI and PIspS transformed cell line, 73 μg/L/hr for the PIDI, PIspS, and DXS transformed cell line, and 142 μg/L/hr for the PIDI, PIspS, DXS, and HDR transformed cell line.

### Example 3

### Methods

Isoprene synthase (PIspS) cloned from Populus trichocarpa and E. coli MEP pathway genes that had been synthesized with unwanted restriction sites removed were cloned into Duet-system expression vectors (Novagen). These constructs were transformed into ScarabXpress® T7lac E. coli in different combinations. Previous testing had revealed that optimal isoprene production was obtained with the genes in the following vectors: DXS (1)-COLADuet™ (O); DXR (2)-pCDFDuet™ (D); CMS and MCS (3)-D; CMK (4)-pACYCDuet™ (A); HDS (6)-O; HDR (7)-A; IDI (8)-pETDuet™ (E); PIspS (9)-E.

Isoprene Measurement

Isoprene productivity of bacterial cultures in sealed vials and fermenter by GC-MSD headspace analysis.

To detect isoprene, a 0.5-mL gas sample from the headspace air above the liquid culture was sampled using a syringe and analyzed on an analytical gas chromatograph with a mass sensitive detector (models 7890 and 5970, Agilent technologies, Santa Clara, Calif., USA). The chromatography was achieved on a 30 m fused silica column (model HP-5MS, Agilent technologies) with a 20 mL min⁻¹ flow of high-purity helium carrier gas at 65° C. isothermal oven temperature.

Isoprene calibrations were made by mixing a standard curve in a multi-stage dilution. A high standard was made by adding 5 μL of liquid isoprene (Fluka Chemical, St. Louis, Mo., USA) in a 1 L gas-mixing bulb filled with N₂ gas. Low standards ranged from 1.3 μg L⁻¹ to 3405 μg L⁻¹.

Isoprene Production in Sealed Vials

Transformed cells were grown in Luria Broth (LB) in sealed vials with appropriate antibiotics and with 0.6 mM IPTG added to induce expression of the introduced genes. The headspace over the cell cultures was assayed for isoprene content by GC-MSD and cells from parallel flask-grown cultures were collected to obtain samples for mRNA quantitation, every three hours for nine hours. The optical densities of the vial-grown cultures were also assayed after nine hours.

Isoprene Production in a Flow-Through Fermenter

The inventors analyzed the isoprene production from pure glucose in a controlled fermenter culture. All experiments were performed with O1E89, which over expresses DXS, IDI and IspS under induction of IPTG (0.6 mM was used in this experiment). Kanamycin and ampicillin (50 μg/ml) were added to the medium to maintain two expression vectors in the bacteria. The fermentation medium was 20 g L⁻¹ glucose in K12 salts medium. Overnight cultures were started in 50 ml LB medium at 37° C. and 50 μg/L ampicillin and kanamycin were included in the medium. No IPTG was added for overnight incubation.

To start the fermenter culture, glucose was autoclaved in 100 ml water separately from the medium. 350 ml K12 medium were autoclaved in the fermenter. After cooling down, glucose solution was added to the fermenter and kanamycin, ampicillin, and IPTG were added to make the final concentrations reach 0.5 μg/L, 0.5 μg/L and 0.6 mM, respectively, for a 500 ml of medium. After the medium temperature reached 37° C., overnight growth LB medium was added and isoprene fermentation was started with continuous filtered air sparging (0.05 L h⁻¹), and the medium pH was maintained at 7. Fermenter headspace air was sampled (0.5 ml) every hour for isoprene analysis using GC-MSD, and, at the same time, a 1-ml medium sample was filtered for later glucose concentration analysis.

The source of glucose for fermentation can be laboratory-grade glucose, acid-hydrolyzed cellulose, enzymatically hydrolyzed cellulose or washed pulp mill sludge hydrolyzed by enzymes or acid hydrolysis.

The isoprene production rate was calculated by multiplying the isoprene concentration in the fermenter headspace by the flow rate and scaling to a 1 L standard culture size according to Equation 1.

Isoprene production=[(μg isoprene/L air)*(air flow L/hour)]/L culture. (Equation 1)

The inventors calculated the efficiency of glucose conversion into isoprene as percent carbon yield (% CY) by dividing the moles of carbon in the isoprene produced by the moles of carbon in the carbon source (such as the moles of glucose carbon in the fermentation medium). This number is multiplied by 100% to give a percentage value as shown in Equation 2.

% CY=(mol carbon in isoprene produced)/(mol carbon in carbon source)*100. (Equation 2)

Results

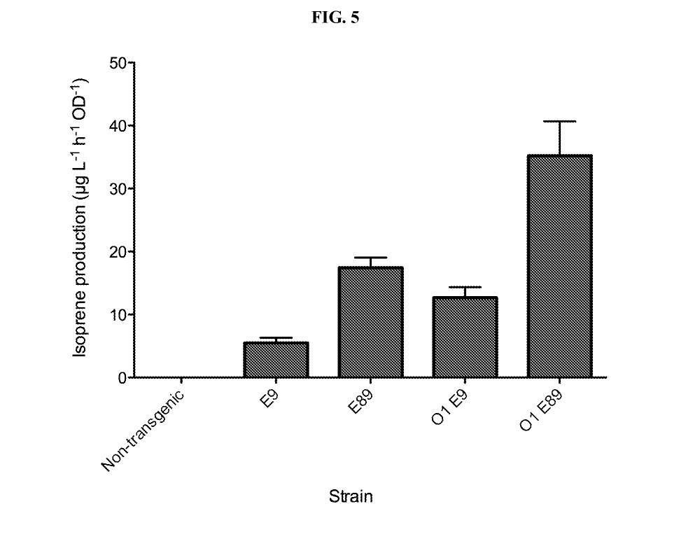

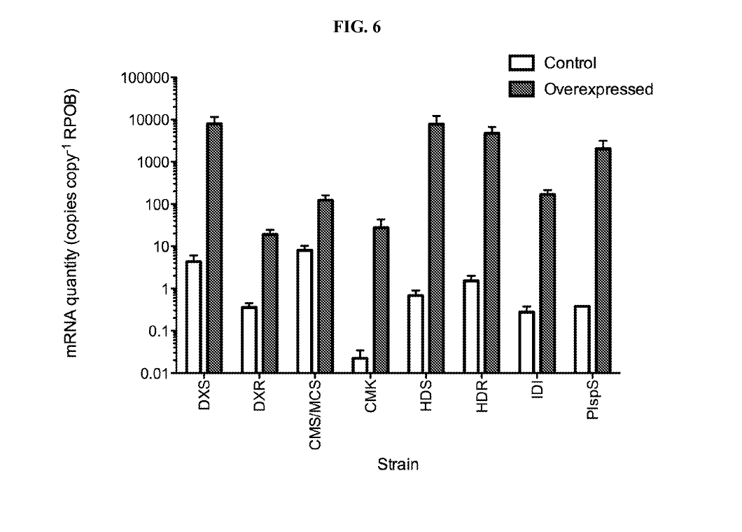

Isoprene production increased as PIspS, IDI, and DXS were added sequentially, and all three of these genes were needed for highest isoprene production (FIG. 5). Therefore, these genes were included in every other transgenic line that was tested, with all 31 possible combinations of the other six genes, to find the lines with optimal isoprene production. Overexpression of the genes was successful, with greater accumulation of each gene's mRNA in cell lines with the gene overexpressed than in lines in which it was not overexpressed (FIG. 6).

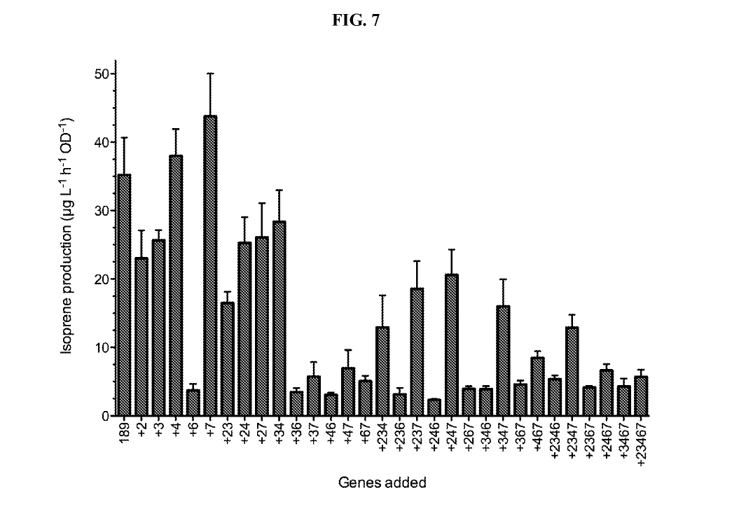

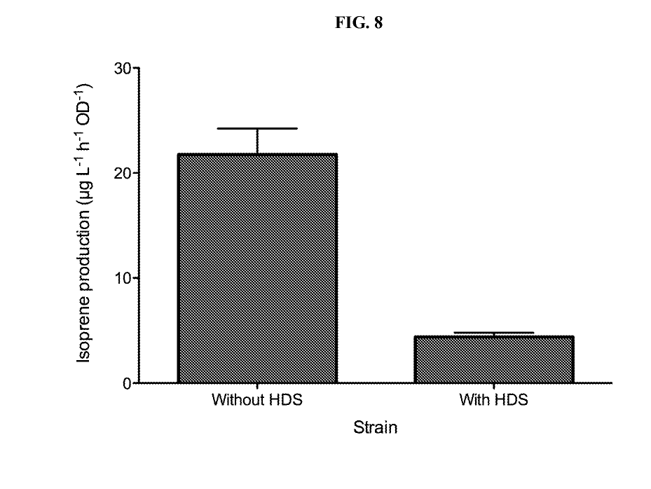

This overexpression did not necessarily lead to increases in isoprene production as more genes were added. When CMK and HDR were added to the standard DXS/IDI/IspS combination of overexpressed genes, isoprene production remained constant and increased in some trials, but in all other cases, emission decreased when additional genes were overexpressed (FIG. 7). The clearest pattern that emerged was seen in HDS overexpression; when lines that did overexpress HDS were compared to their counterparts that did not, isoprene production almost invariably decreased (FIG. 8). In general, there was less isoprene production by cell lines that overexpressed more genes, but the extent of this decrease depended partially on which genes were overexpressed and the vectors in which they were overexpressed.

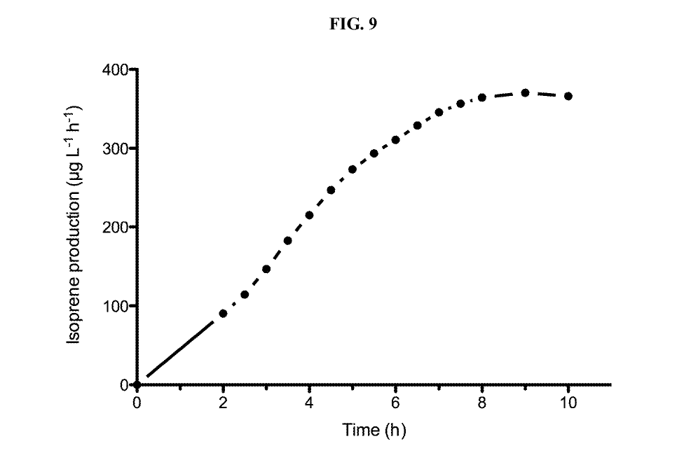

Isoprene production rate in the fermenter culture was higher. In this example (FIG. 9) isoprene was produced at pH 7 with an airflow rate of 3 L h⁻¹. In this example, the maximum isoprene production rate was 370 μg isoprene L⁻¹ culture h⁻¹. The culture consumed 16.15 g glucose in 10 h, and the total isoprene produced was 2.438×10⁻³ g. The percent conversion of carbon into isoprene is calculated as:

% CY=(2.438×10⁻³ g isoprene*1/68.1 mol/g*5 C/mol)/[(16.15 g glucose*1/180 mol/g*6 C/mol)]*100%=0.033%.

This example demonstrates isoprene production with one bacterial strain under one set of conditions. Manipulating oxygen levels, airflow rate, pH, and temperature can optimize the fermentation conditions. Balancing the expression of the necessary genes, reducing the need for antibiotics in the culture, and using a different host organism other than E. coli will allow for improved isoprene production rate and carbon yield.

### Example 4

mRNA accumulation for cell lines overexpressing or not overexpressing MEP pathway genes and PIspS.

RNA polymerase B (RPOB) was used as the control gene. Cell cultures were started and incubated as described in Example 4. Overnight cultures were started as in Example 4, and then an 8-mL aliquot of each line was mixed with 10 mL sterile LB containing appropriate antibiotics and 0.6 mM IPTG in sterile 50-mL Erlenmeyer flasks. These vessels were then shaken at 37° C. and 150 rpm for nine hours, with 1.5-mL samples taken to collect cells for mRNA analyses. Cells were pelleted by centrifugation in a tabletop microfuge at maximum speed for 10 min. Total RNA was extracted from frozen cell pellets using the RNeasy Mini Kit (Qiagen Inc.), according to the manufacturer's instructions. Quantitative polymerase chain reaction (QPCR) was then carried out on 0.25 μg of total RNA per gene analyzed as detailed in the methods section of Example 3.

### Example 5

Isoprene production by cell lines overexpressing different combinations of MEP pathway genes with PIspS was measured in sealed vials. Overnight cultures of the genotype-verified cell lines were grown at 37° C. and 200 rpm in LB media with appropriate antibiotics. For sampling, a 2-mL aliquot of each line was mixed with 3 mL sterile LB containing appropriate antibiotics and 0.6 mM IPTG in sterile 20-mL screw-cap vials. These vessels were then shaken at 37° C. and 150 rpm for nine hours, with samples measured at the end of a 9-hr incubation. Isoprene production was determined by GC-MSD analysis of 0.5-mL samples from the headspace of the screw-cap vials and compared with a 10-point standard curve made by diluting liquid isoprene in N₂ gas. Isoprene production is shown in FIG. 7.

### Example 6

Isoprene production in a 1.3 L fermenter culture with strain O1E89 was measured. An overnight culture was started with 50 ml LB medium inoculated with O1E89 strain at 37° C. with 50 ug/L ampicillin and kanamycin. The fermenter vessel was autoclaved with 350 ml K12 medium. After cooling down, a 100-mL sterile glucose solution was added to the fermentor to reach 20 g L⁻¹ and more kanamycin, ampicillin, and IPTG were added to make the final concentrations reach 0.5 μg/L, 0.5 μg/L and 0.6 mM, respectively for 500 ml medium. After the medium temperature reached 37° C., the 50-mL overnight culture was added and isoprene fermentation was started with continuous sparging of filtered air at 0.05 L min⁻¹. Medium pH was maintained at 7 by addition of 0.1 M NaOH as needed. Fermenter headspace air was sampled for isoprene analysis every 0.5 hours with a 1-mL syringes and isoprene was quantified by GC-MSD by comparing with a 10-point standard curve made by diluting liquid isoprene in N₂ gas. Isoprene production is shown in FIG. 9.

### Example 7

Isoprene was produced following the methods of Example 3. The fermentation medium was pulp mill sludge. The inventors obtained pulp mill sludge from a Kraft pulp mill in Wisconsin. Pulp mill sludge is made up of concentrated solids from a variety of waste streams in a mill. The majority of the solids consist of pulp fibers, woody debris, paper additives, and residual pulping chemicals. The inventors sent a sample to a water chemistry laboratory for analysis of inorganic materials in the sludge. Results are shown in Table 1.

TABLE 1
Chemical analysis of pulp mill sludge.

| | Content | |
|---|---|---|
| Element | (mg L⁻¹) | Method |
| As | <4 | EPA 200.7 |
| Ca | 23,700 | EPA 200.7 |
| Cu | 131 | EPA 200.7 |
| Fe | 4098 | EPA 200.7 |
| (dissolved) | | |
| K | 1100 | EPA 200.7 |
| Mg | 3400 | EPA 200.7 |
| Mn | 261 | EPA 200.7 |
| (dissolved) | | |
| Na | 500 | EPA 200.7 |
| P | 2691 | 4500 P F |
| Pb | 17 | EPA 200.7 |
| SO₄⁻ | 14,890 | EPA 200.7 |
| Zn | 261 | EPA 200.7 |

To measure total sugar content of pulp mill sludge, the inventors hydrolyzed 0.7±0.01 g of oven-dry sludge using 3 mL of 72% H₂SO₄ for 1 hour in a 30° C. water bath. Samples were diluted to 87 mL and autoclaved for 1 hour. Samples were diluted and analyzed by ion chromatography (Model ICS3000, Dionex corp.) using an internal standard of 0.2 mg mL⁻¹ myo-inositol. Total available sludge sugars are shown in Table 2.

TABLE 2
Major components of hydrolyzed sludge (wt %) analyzed by ion
chromatography after acid hydrolysis.

| | Glucan | Araban | Galactan | Xylan | Mannan | Total |
|---|---|---|---|---|---|---|
| Acid hydrolysis | 40.78 | 0.09 | 0.09 | 5.59 | 1.96 | 48.51 |

Pulp mill sludge was hydrolyzed enzymatically with commercial enzymes (Cellic Ctec®, Novozymes, Inc., and Accelerase® 1500, Genencor, Inc.) following manufacturer's recommended procedures designed for cellulose. Total mass conversion was calculated by filtering the resultant mixture and weighing the remaining solids. Sugar conversion efficiency is reported as the ratio of mass conversion by enzymes to mass conversion by acid hydrolysis and is shown in Table 3.

TABLE 3
Conversion of sludge to soluble sugars by enzymatic
hydrolysis. Sugar conversion efficiency is based on the ratio of mass
conversion by enzymatic hydrolysis to mass conversion by acid hydrolysis.

| | Initial | Final | Sugar conversion |
|---|---|---|---|
| | mass | mass | efficiency (%) |
| Accelerase ® 1500 (Washed sludge) | 50.88 | 35.32 | 62.9 |
| Accelerase ® 1500 (Unwashed) | 50.26 | 32.31 | 73.5 |
| Cellic Ctec ® (Unwashed) | 50.54 | 35.80 | 60.0 |

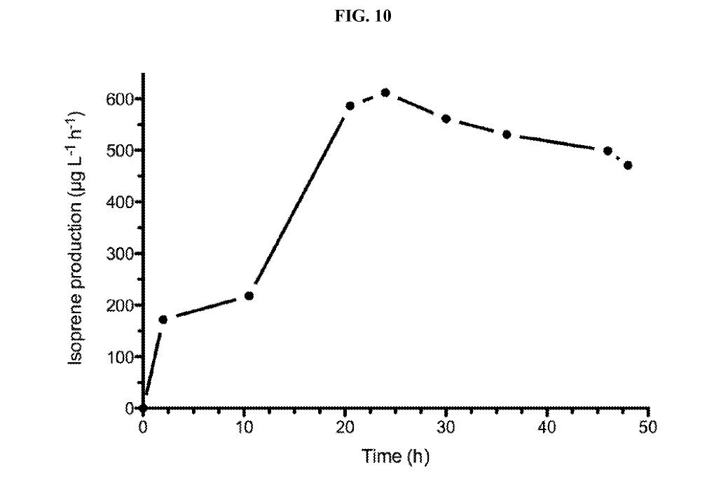

Fermentation of sludge to isoprene was carried out using a method similar to that in Example 3. Isoprene production from hydrolyzed pulp mill sludge in a 1.3 L fermenter culture with strain A17E89 (containing DXS, HDR, poplar IDI and poplar IspS) was measured. An overnight culture was started with 5-ml LB medium inoculated with A17E89 strain at 37° C. with 50 μg/L ampicillin and 35 μg/mL chloramphenicol. The fermenter vessel was autoclaved with 500-ml M9 medium. After cooling down, a 200-mL sterile sludge hydrosylate solution was added to the fermentor and more chloramphenicol, ampicillin and IPTG were added to make the final concentrations reach 0.5 μg/L, 0.35 μg/L and 0.6 mM, respectively, for 500 ml of medium. After the medium temperature reached 37° C., the 50-mL overnight culture was added and isoprene fermentation was started with continuous sparging of filtered air at 0.311 L min⁻¹. Medium pH was maintained at 7 by addition of 0.1 M NaOH as needed. Fermenter headspace air was sampled for isoprene analysis with a 0.5 mL syringes and isoprene was quantified by GC-MSD by comparing with a 10-point standard curve made by diluting liquid isoprene in N₂ gas. Isoprene production is shown in FIG. 10.

### Example 8

### Prophetic Plan for Operon Use

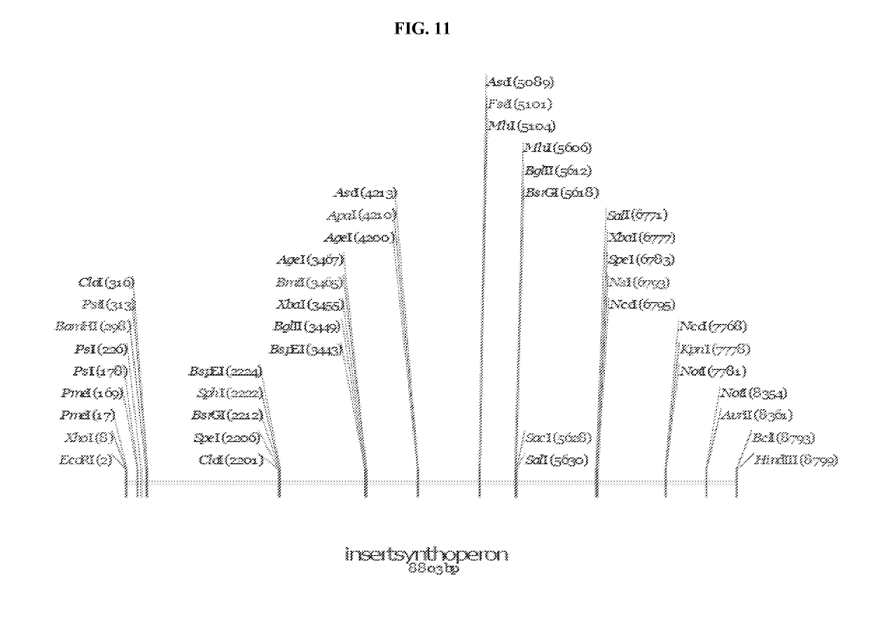

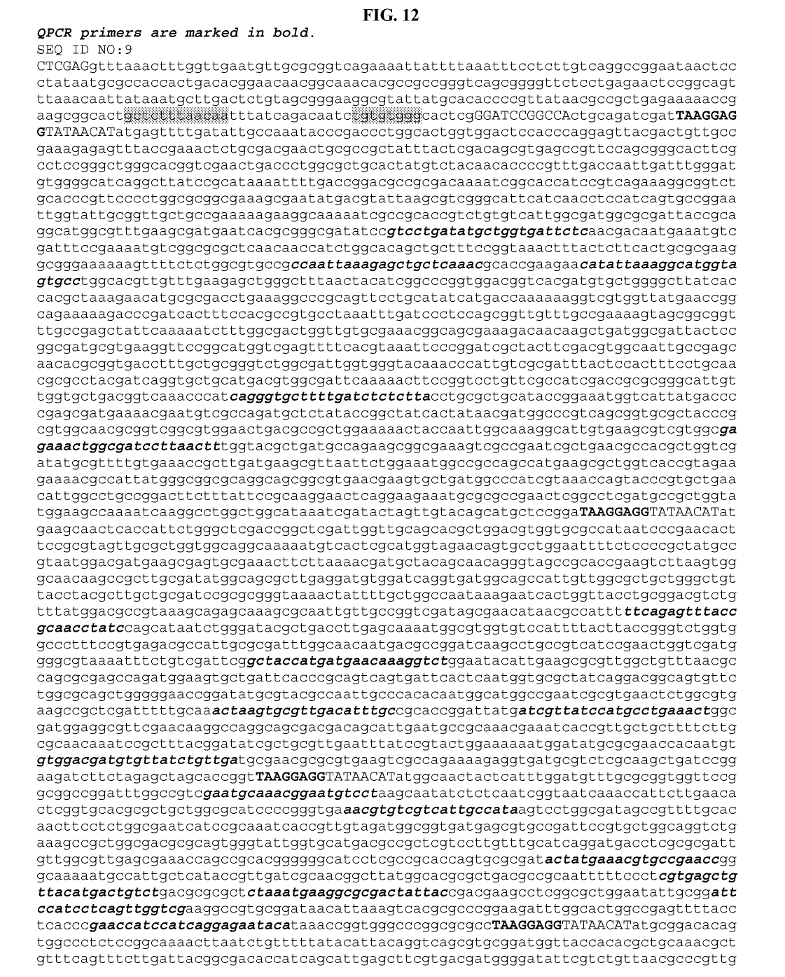

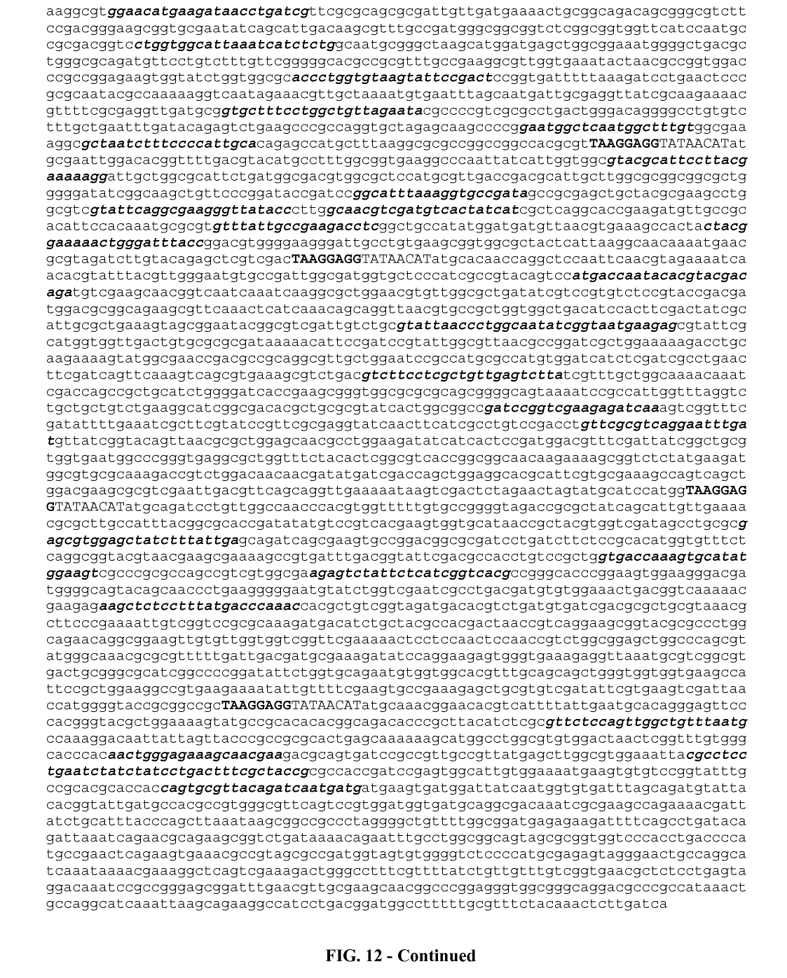

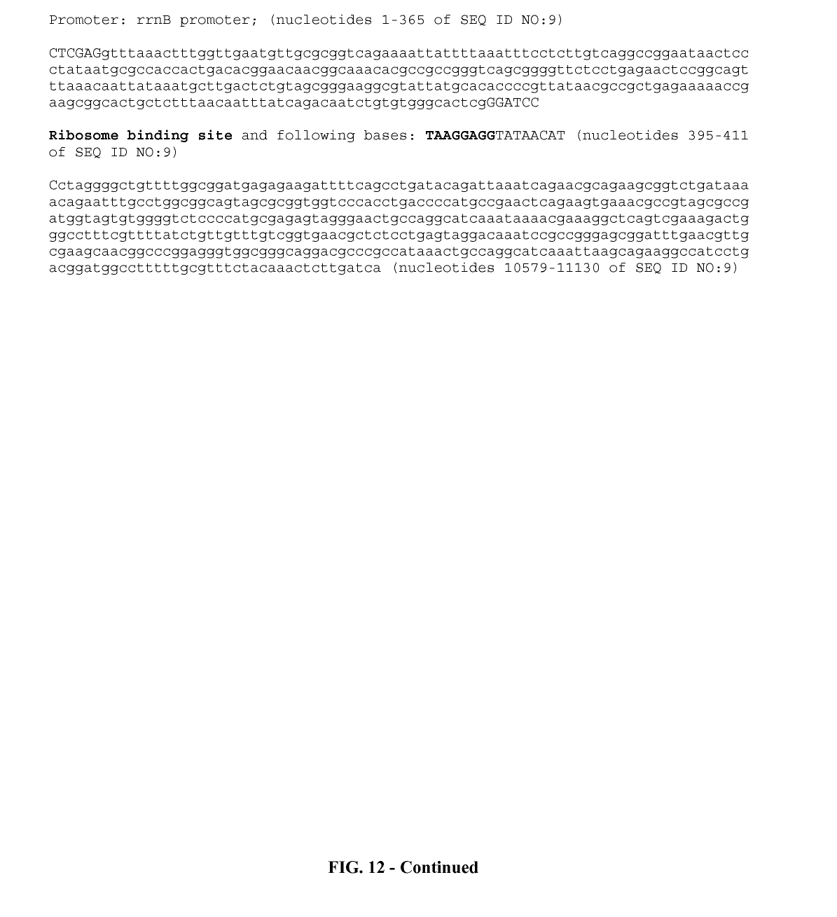

The inventors designed and constructed a synthetic operon that will allow the testing of different combinations of genes as well as individual control of the genes by different promoters. The synthetic operon insert is described in FIG. 11. The sequence for the synthetic operon insert is shown in FIG. 12.

The synthetic operon contains the coding sequences of the entire E. coli MEP pathway enzymes with restriction sites for enzymes commonly used in cloning removed by introduction of silent mutations. Restriction sites flank each gene and several sets of genes, so that each gene can be removed singly or in combination, to yield a gene combination optimal for isoprene production. In addition, a unique restriction site is present upstream of each gene; this allows introduction of gene-specific promoters and terminators, so that the expression of each gene can be fine-tuned as necessary. The poplar IspS gene can be introduced as well. By PCR amplification with appropriate primers, or by restriction digestion out of a vector, the operon can be cloned into virtually any expression vector or BAC for propagation and expression in any of a number of microbial hosts.

In one case this can be inserted into E. coli or other prokaryote organism using a commercial Bacterial Artificial Chromosome.

In one case this can be inserted into a prokaryotic organism using a linear cloning vector, such as pJazz from Lucigen Corporation (Madison, Wis., USA).

In one case this can be incorporated into the genome of a cyanobacterium such as anabaena, Synecococcus, or Synechocystis using a shuttle vector system.

We claim:

1. A microorganism, wherein the microorganism comprises:
a transgene encoding isopentenyl diphosphate isomerase (IDI);
a transgene encoding isoprene synthase (IspS); and
a transgene encoding 1-deoxy-D-xylulose-5-phosphate synthase (DXS), wherein the microorganism produces isoprene, and wherein the isoprene production averaged for at least 8 hours of growth is at the rate of at least 3 μg/L/hr and at a rate of at least 450 μg/L/hr after 45 hours' production.

2. The microorganism of claim 1, wherein the microorganism is selected from the group consisting of Synechococcus, Synechocystis PCC6803, Thermosynechococcus elongatus BP-1, Synechococcus sp. PCC 7002, Anabaena variablis ATCC29413, Anabaena PCC7120, Nostoc punctiforme ATCC7312, Saccharomyces spp., Pichia spp., Chlamydomonas spp., Anabaena spp., Spirochaeta americana, Thermus thermophilus, Deinococcus radiodurans, Thermus aquaticus, Chloroflexus aurantiacus, Pyrococcus furiosus, Pyrococcus furiosus, Pyrolobus fumarii, natural and modified E. coli, Bacillus spp., Lactobacillus spp., Geobacillus sterothermophilus strain G1.13, Mycobacterium smegmatis, and Clostridia spp.

3. The microorganism of claim 1, wherein the microorganism further comprises a transgene encoding hydroxymethylbutenyl diphosphate reductase (HDR).

4. The microorganism of claim 1, wherein the host transgenic microorganism further comprises a transgene encoding 1-deoxy-D-xylulose-5-phosphate reductoisomerase (DXR).

5. The microorganism of claim 1, wherein the host transgenic microorganism further comprises at least one transgene selected from the group consisting of transgenes encoding 4-diphosphocytidyl-2-C-methyl-D-erythritol synthase (CMS), 4-diphosphocytidyl-2-C-methyl-D-erythritol kinase (CMK), and 2-C-methyl-D-erythritol 2,4-cyclodiphosphate synthase (MCS).

6. The microorganism of claim 1, wherein the microorganism additionally comprises flavodoxin and flavodoxin reductase.

7. The microorganism of claim 1, wherein the microorganism is an E. coli.

8. The microorganism of claim 1, wherein the microorganism is a photosynthetic cyanobacterium.

9. The microorganism of claim 1, wherein at least one of the transgenes is isolated from Populus trichocarpa.

10. The microorganism of claim 9, wherein the transgene is Populus trichocarpa IDI.

11. The microorganism of claim 9 wherein the transgene is Populus trichocarpa IspS.

12. The microorganism of claim 1, wherein the microorganism is Bacillus coagulans.

13. The microorganism of claim 1, wherein the host transgenic microorganism is Synechococcus.

14. The microorganism of claim 1, wherein at least one of the transgenes is isolated from a non-E. coli source and where the transgene has been codon amplified for insertion into an E. coli host transgenic microorganism.

15. The microorganism of claim 1, where the isoprene production from the microorganism averaged for at least 8 hours of growth is at the rate of at least 70 μg/L/h r.

16. The microorganism of claim 1, wherein the isoprene production from the microorganism retains at least 75% of its maximal production rate after 45 hours' production.

17. The microorganism of claim 1, wherein the isoprene production from the microorganism is in a fermentation medium.

18. The microorganism of claim 17, wherein the fermentation medium comprises glucose.

19. The microorganism of claim 17, wherein the fermentation medium comprises paper mill sludge hydrolysate produced by enzyme or acid-catalyzed hydrolysis of waste fibers from a pulp mill.

20. The microorganism of claim 1, wherein the isoprene is recovered.

21. The microorganism of claim 20, wherein the recovered isoprene is modified into the group selected from dimer (10-carbon) hydrocarbons, trimer (15-carbon) hydrocarbons, and mixtures of dimer and trimer hydrocarbons.

22. The microorganism of claim 21, wherein the dimer and/or trimer hydrocarbons are hydrogenated to long-chain, branched alkanes suitable for use in fuel or solvents.

23. The microorganism of claim 21, wherein the dimer hydrocarbons are used in organosolv pulping.

24. The microorganism of claim 1, wherein the isoprene is used to produce rubber.

25. The microorganism of claim 1, wherein the isoprene is polymerized with catalyst systems to form homopolymers of cis-3-polyisoprene.

26. The microorganism of claim 1, wherein the isoprene is co-polymerized with styrene or butadiene to produce an elastomer.

27. The microorganism of claim 1, wherein the isoprene is polymerized with an oxidant to form hydroxyl-terminated polyisoprene.

28. The microorganism of claim 27, wherein the oxidant is hydrogen peroxide.

29. The microorganism of claim 1, wherein the isoprene is polymerized into liquid fuels that are infrastructure compatible with current gasoline, diesel or jet engines.


SEQUENCE LISTING (9 sequences)

SEQ ID NO: 1 - DNA, 1863 bp, Escherichia coli
atgagttttgatattgccaaatacccgaccctggcactggtggactccacccaggagttacgactgttgccgaaagagagtttaccgaaactctgcgacgaactgcgccgctatttactcgacagcgtgagccgttccagcgggcacttcgcctccgggctgggcacggtcgaactgaccctggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccctggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaaactttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctacgatcaggtgctgcatgacgtggcgattcaaaaacttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataa

SEQ ID NO: 2 - DNA, 1197 bp, Escherichia coli
atgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattttctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgtggatcaggtgatggcagccattgttggcgctgctgggctgttacctacgcttgctgcgatccgcgcgggtaaaactattttgctggccaataaagaatcactggttacctgcggacgtctgtttatggacgccgtaaagcagagcaaagcgcaattgttgccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgcgagccagatggaagtgctgattcacccgcagtcagtgattcactcaatggtgcgctatcaggacggcagtgttctggcgcagctgggggaaccggatatgcgtacgccaattgcccacacaatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctga

SEQ ID NO: 3 - DNA, 711 bp, Escherichia coli
atggcaactactcatttggatgtttgcgcggtggttccggcggccggatttggccgtcgaatgcaaacggaatgtcctaagcaatatctctcaatcggtaatcaaaccattcttgaacactcggtgcacgcgctgctggcgcatccccgggtgaaacgtgtcgtcattgccataagtcctggcgatagccgttttgcacaacttcctctggcgaatcatccgcaaatcaccgttgtagatggcggtgatgagcgtgccgattccgtgctggcaggtctgaaagccgctggcgacgcgcagtgggtattggtgcatgacgccgctcgtccttgtttgcatcaggatgacctcgcgcgattgttggcgttgagcgaaaccagccgcacggggggcatcctcgccgcaccagtgcgcgatactatgaaacgtgccgaaccgggcaaaaatgccattgctcataccgttgatcgcaacggcttatggcacgcgctgacgccgcaatttttccctcgtgagctgttacatgactgtctgacgcgcgctctaaatgaaggcgcgactattaccgacgaagcctcggcgctggaatattgcggattccatcctcagttggtcgaaggccgtgcggataacattaaagtcacgcgcccggaagatttggcactggccgagttttacctcacccgaaccatccatcaggagaatacataa

SEQ ID NO: 4 - DNA, 852 bp, Escherichia coli
atgcggacacagtggccctctccggcaaaacttaatctgtttttatacattacaggtcagcgtgcggatggttaccacacgctgcaaacgctgtttcagtttcttgattacggcgacaccatcagcattgagcttcgtgacgatggggatattcgtctgttaacgcccgttgaaggcgtggaacatgaagataacctgatcgttcgcgcagcgcgattgttgatgaaaactgcggcagacagcgggcgtcttccgacgggaagcggtgcgaatatcagcattgacaagcgtttgccgatgggcggcggtctcggcggtggttcatccaatgccgcgacggtcctggtggcattaaatcatctctggcaatgcgggctaagcatggatgagctggcggaaatggggctgacgctgggcgcagatgttcctgtctttgttcgggggcacgccgcgtttgccgaaggcgttggtgaaatactaacgccggtggacccgccggagaagtggtatctggtggcgcaccctggtgtaagtattccgactccggtgatttttaaagatcctgaactcccgcgcaatacgccaaaaaggtcaatagaaacgttgctaaaatgtgaatttagcaatgattgcgaggttatcgcaagaaaacgttttcgcgaggttgatgcggtgctttcctggctgttagaatacgccccgtcgcgcctgactgggacaggggcctgtgtctttgctgaatttgatacagagtctgaagcccgccaggtgctagagcaagccccggaatggctcaatggctttgtggcgaaaggcgctaatctttccccattgcacagagccatgctttaa

SEQ ID NO: 5 - DNA, 480 bp, Escherichia coli
atgcgaattggacacggttttgacgtacatgcctttggcggtgaaggcccaattatcattggtggcgtacgcattccttacgaaaaaggattgctggcgcattctgatggcgacgtggcgctccatgcgttgaccgacgcattgcttggcgcggcggcgctgggggatatcggcaagctgttcccggataccgatccggcatttaaaggtgccgatagccgcgagctgctacgcgaagcctggcgtcgtattcaggcgaagggttatacccttggcaacgtcgatgtcactatcatcgctcaggcaccgaagatgttgccgcacattccacaaatgcgcgtgtttattgccgaagacctcggctgccatatggatgatgttaacgtgaaagccactactacggaaaaactgggatttaccggacgtggggaagggattgcctgtgaagcggtggcgctactcattaaggcaacaaaatga

SEQ ID NO: 6 - DNA, 1119 bp, Escherichia coli
atgcacaaccaggctccaattcaacgtagaaaatcaacacgtatttacgttgggaatgtgccgattggcgatggtgctcccatcgccgtacagtccatgaccaatacacgtacgacagatgtcgaagcaacggtcaatcaaatcaaggcgctggaacgtgttggcgctgatatcgtccgtgtctccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagacctgcaagaaaagtatggcgaaccgacgccgcaggcgttgctggaatccgccatgcgccatgtggatcatctcgatcgcctgaacttcgatcagttcaaagtcagcgtgaaagcgtctgacgtcttcctcgctgttgagtcttatcgtttgctggcaaaacaaatcgaccagccgctgcatctggggatcaccgaagcgggtggcgcgcgcagcggggcagtaaaatccgccattggtttaggtctgctgctgtctgaaggcatcggcgacacgctgcgcgtatcactggcggccgatccggtcgaagagatcaaagtcggtttcgatattttgaaatcgcttcgtatccgttcgcgaggtatcaacttcatcgcctgtccgacctgttcgcgtcaggaatttgatgttatcggtacagttaacgcgctggagcaacgcctggaagatatcatcactccgatggacgtttcgattatcggctgcgtggtgaatggcccgggtgaggcgctggtttctacactcggcgtcaccggcggcaacaagaaaagcggtctctatgaagatggcgtgcgcaaagaccgtctggacaacaacgatatgatcgaccagctggaggcacgcattcgtgcgaaagccagtcagctggacgaagcgcgtcgaattgacgttcagcaggttgaaaaataa

SEQ ID NO: 7 - DNA, 951 bp, Escherichia coli
atgcagatcctgttggccaacccacgtggtttttgtgccggggtagaccgcgctatcagcattgttgaaaacgcgcttgccatttacggcgcaccgatatatgtccgtcacgaagtggtgcataaccgctacgtggtcgatagcctgcgcgagcgtggagctatctttattgagcagatcagcgaagtgccggacggcgcgatcctgatcttctccgcacatggtgtttctcaggcggtacgtaacgaagcgaaaagccgtgatttgacggtattcgacgccacctgtccgctggtgaccaaagtgcatatggaagtcgcccgcgccagccgtcgtggcgaagagtctattctcatcggtcacgccgggcacccggaagtggaagggacgatggggcagtacagcaaccctgaagggggaatgtatctggtcgaatcgcctgacgatgtgtggaaactgacggtcaaaaacgaagagaagctctcctttatgacccaaaccacgctgtcggtagatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagatatccaggaagagtgggtgaaagaggttaaatgcgtcggcgtgactgcgggcgcatcggccccggatattctggtgcagaatgtggtggcacgtttgcagcagctgggtggtggtgaagccattccgctggaaggccgtgaagaaaatattgttttcgaagtgccgaaagagctgcgtgtcgatattcgtgaagtcgattaa

SEQ ID NO: 8 - DNA, 549 bp, Escherichia coli
atgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataa

SEQ ID NO: 9 - DNA, 8791 bp, Artificial Sequence
  Synthetic operon
ctcgaggtttaaactttggttgaatgttgcgcggtcagaaaattattttaaatttcctcttgtcaggccggaataactccctataatgcgccaccactgacacggaacaacggcaaacacgccgccgggtcagcggggttctcctgagaactccggcagtttaaacaattataaatgcttgactctgtagcgggaaggcgtattatgcacaccccgttataacgccgctgagaaaaaccgaagcggcactgctctttaacaatttatcagacaatctgtgtgggcactcgggatccggccactgcagatcgattaaggaggtataacatatgagttttgatattgccaaatacccgaccctggcactggtggactccacccaggagttacgactgttgccgaaagagagtttaccgaaactctgcgacgaactgcgccgctatttactcgacagcgtgagccgttccagcgggcacttcgcctccgggctgggcacggtcgaactgaccctggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccctggcgcggcgaaagcgaatatgacgtattaagcgtcgggcattcatcaacctccatcagtgccggaattggtattgcggttgctgccgaaaaagaaggcaaaaatcgccgcaccgtctgtgtcattggcgatggcgcgattaccgcaggcatggcgtttgaagcgatgaatcacgcgggcgatatccgtcctgatatgctggtgattctcaacgacaatgaaatgtcgatttccgaaaatgtcggcgcgctcaacaaccatctggcacagctgctttccggtaaactttactcttcactgcgcgaaggcgggaaaaaagttttctctggcgtgccgccaattaaagagctgctcaaacgcaccgaagaacatattaaaggcatggtagtgcctggcacgttgtttgaagagctgggctttaactacatcggcccggtggacggtcacgatgtgctggggcttatcaccacgctaaagaacatgcgcgacctgaaaggcccgcagttcctgcatatcatgaccaaaaaaggtcgtggttatgaaccggcagaaaaagacccgatcactttccacgccgtgcctaaatttgatccctccagcggttgtttgccgaaaagtagcggcggtttgccgagctattcaaaaatctttggcgactggttgtgcgaaacggcagcgaaagacaacaagctgatggcgattactccggcgatgcgtgaaggttccggcatggtcgagttttcacgtaaattcccggatcgctacttcgacgtggcaattgccgagcaacacgcggtgacctttgctgcgggtctggcgattggtgggtacaaacccattgtcgcgatttactccactttcctgcaacgcgcctacgatcaggtgctgcatgacgtggcgattcaaaaacttccggtcctgttcgccatcgaccgcgcgggcattgttggtgctgacggtcaaacccatcagggtgcttttgatctctcttacctgcgctgcataccggaaatggtcattatgaccccgagcgatgaaaacgaatgtcgccagatgctctataccggctatcactataacgatggcccgtcagcggtgcgctacccgcgtggcaacgcggtcggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccgccagccatgaagcgctggtcaccgtagaagaaaacgccattatgggcggcgcaggcagcggcgtgaacgaagtgctgatggcccatcgtaaaccagtacccgtgctgaacattggcctgccggacttctttattccgcaaggaactcaggaagaaatgcgcgccgaactcggcctcgatgccgctggtatggaagccaaaatcaaggcctggctggcataaatcgatactagttgtacagcatgctccggataaggaggtataacatatgaagcaactcaccattctgggctcgaccggctcgattggttgcagcacgctggacgtggtgcgccataatcccgaacacttccgcgtagttgcgctggtggcaggcaaaaatgtcactcgcatggtagaacagtgcctggaattttctccccgctatgccgtaatggacgatgaagcgagtgcgaaacttcttaaaacgatgctacagcaacagggtagccgcaccgaagtcttaagtgggcaacaagccgcttgcgatatggcagcgcttgaggatgtggatcaggtgatggcagccattgttggcgctgctgggctgttacctacgcttgctgcgatccgcgcgggtaaaactattttgctggccaataaagaatcactggttacctgcggacgtctgtttatggacgccgtaaagcagagcaaagcgcaattgttgccggtcgatagcgaacataacgccatttttcagagtttaccgcaacctatccagcataatctgggatacgctgaccttgagcaaaatggcgtggtgtccattttacttaccgggtctggtggccctttccgtgagacgccattgcgcgatttggcaacaatgacgccggatcaagcctgccgtcatccgaactggtcgatggggcgtaaaatttctgtcgattcggctaccatgatgaacaaaggtctggaatacattgaagcgcgttggctgtttaacgccagcgcgagccagatggaagtgctgattcacccgcagtcagtgattcactcaatggtgcgctatcaggacggcagtgttctggcgcagctgggggaaccggatatgcgtacgccaattgcccacacaatggcatggccgaatcgcgtgaactctggcgtgaagccgctcgatttttgcaaactaagtgcgttgacatttgccgcaccggattatgatcgttatccatgcctgaaactggcgatggaggcgttcgaacaaggccaggcagcgacgacagcattgaatgccgcaaacgaaatcaccgttgctgcttttcttgcgcaacaaatccgctttacggatatcgctgcgttgaatttatccgtactggaaaaaatggatatgcgcgaaccacaatgtgtggacgatgtgttatctgttgatgcgaacgcgcgtgaagtcgccagaaaagaggtgatgcgtctcgcaagctgatccggaagatcttctagagctagcaccggttaaggaggtataacatatggcaactactcatttggatgtttgcgcggtggttccggcggccggatttggccgtcgaatgcaaacggaatgtcctaagcaatatctctcaatcggtaatcaaaccattcttgaacactcggtgcacgcgctgctggcgcatccccgggtgaaacgtgtcgtcattgccataagtcctggcgatagccgttttgcacaacttcctctggcgaatcatccgcaaatcaccgttgtagatggcggtgatgagcgtgccgattccgtgctggcaggtctgaaagccgctggcgacgcgcagtgggtattggtgcatgacgccgctcgtccttgtttgcatcaggatgacctcgcgcgattgttggcgttgagcgaaaccagccgcacggggggcatcctcgccgcaccagtgcgcgatactatgaaacgtgccgaaccgggcaaaaatgccattgctcataccgttgatcgcaacggcttatggcacgcgctgacgccgcaatttttccctcgtgagctgttacatgactgtctgacgcgcgctctaaatgaaggcgcgactattaccgacgaagcctcggcgctggaatattgcggattccatcctcagttggtcgaaggccgtgcggataacattaaagtcacgcgcccggaagatttggcactggccgagttttacctcacccgaaccatccatcaggagaatacataaaccggtgggcccggcgcgcctaaggaggtataacatatgcggacacagtggccctctccggcaaaacttaatctgtttttatacattacaggtcagcgtgcggatggttaccacacgctgcaaacgctgtttcagtttcttgattacggcgacaccatcagcattgagcttcgtgacgatggggatattcgtctgttaacgcccgttgaaggcgtggaacatgaagataacctgatcgttcgcgcagcgcgattgttgatgaaaactgcggcagacagcgggcgtcttccgacgggaagcggtgcgaatatcagcattgacaagcgtttgccgatgggcggcggtctcggcggtggttcatccaatgccgcgacggtcctggtggcattaaatcatctctggcaatgcgggctaagcatggatgagctggcggaaatggggctgacgctgggcgcagatgttcctgtctttgttcgggggcacgccgcgtttgccgaaggcgttggtgaaatactaacgccggtggacccgccggagaagtggtatctggtggcgcaccctggtgtaagtattccgactccggtgatttttaaagatcctgaactcccgcgcaatacgccaaaaaggtcaatagaaacgttgctaaaatgtgaatttagcaatgattgcgaggttatcgcaagaaaacgttttcgcgaggttgatgcggtgctttcctggctgttagaatacgccccgtcgcgcctgactgggacaggggcctgtgtctttgctgaatttgatacagagtctgaagcccgccaggtgctagagcaagccccggaatggctcaatggctttgtggcgaaaggcgctaatctttccccattgcacagagccatgctttaaggcgcgccggccggccacgcgttaaggaggtataacatatgcgaattggacacggttttgacgtacatgcctttggcggtgaaggcccaattatcattggtggcgtacgcattccttacgaaaaaggattgctggcgcattctgatggcgacgtggcgctccatgcgttgaccgacgcattgcttggcgcggcggcgctgggggatatcggcaagctgttcccggataccgatccggcatttaaaggtgccgatagccgcgagctgctacgcgaagcctggcgtcgtattcaggcgaagggttatacccttggcaacgtcgatgtcactatcatcgctcaggcaccgaagatgttgccgcacattccacaaatgcgcgtgtttattgccgaagacctcggctgccatatggatgatgttaacgtgaaagccactactacggaaaaactgggatttaccggacgtggggaagggattgcctgtgaagcggtggcgctactcattaaggcaacaaaatgaacgcgtagatcttgtacagagctcgtcgactaaggaggtataacatatgcacaaccaggctccaattcaacgtagaaaatcaacacgtatttacgttgggaatgtgccgattggcgatggtgctcccatcgccgtacagtccatgaccaatacacgtacgacagatgtcgaagcaacggtcaatcaaatcaaggcgctggaacgtgttggcgctgatatcgtccgtgtctccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagacctgcaagaaaagtatggcgaaccgacgccgcaggcgttgctggaatccgccatgcgccatgtggatcatctcgatcgcctgaacttcgatcagttcaaagtcagcgtgaaagcgtctgacgtcttcctcgctgttgagtcttatcgtttgctggcaaaacaaatcgaccagccgctgcatctggggatcaccgaagcgggtggcgcgcgcagcggggcagtaaaatccgccattggtttaggtctgctgctgtctgaaggcatcggcgacacgctgcgcgtatcactggcggccgatccggtcgaagagatcaaagtcggtttcgatattttgaaatcgcttcgtatccgttcgcgaggtatcaacttcatcgcctgtccgacctgttcgcgtcaggaatttgatgttatcggtacagttaacgcgctggagcaacgcctggaagatatcatcactccgatggacgtttcgattatcggctgcgtggtgaatggcccgggtgaggcgctggtttctacactcggcgtcaccggcggcaacaagaaaagcggtctctatgaagatggcgtgcgcaaagaccgtctggacaacaacgatatgatcgaccagctggaggcacgcattcgtgcgaaagccagtcagctggacgaagcgcgtcgaattgacgttcagcaggttgaaaaataagtcgactctagaactagtatgcatccatggtaaggaggtataacatatgcagatcctgttggccaacccacgtggtttttgtgccggggtagaccgcgctatcagcattgttgaaaacgcgcttgccatttacggcgcaccgatatatgtccgtcacgaagtggtgcataaccgctacgtggtcgatagcctgcgcgagcgtggagctatctttattgagcagatcagcgaagtgccggacggcgcgatcctgatcttctccgcacatggtgtttctcaggcggtacgtaacgaagcgaaaagccgtgatttgacggtattcgacgccacctgtccgctggtgaccaaagtgcatatggaagtcgcccgcgccagccgtcgtggcgaagagtctattctcatcggtcacgccgggcacccggaagtggaagggacgatggggcagtacagcaaccctgaagggggaatgtatctggtcgaatcgcctgacgatgtgtggaaactgacggtcaaaaacgaagagaagctctcctttatgacccaaaccacgctgtcggtagatgacacgtctgatgtgatcgacgcgctgcgtaaacgcttcccgaaaattgtcggtccgcgcaaagatgacatctgctacgccacgactaaccgtcaggaagcggtacgcgccctggcagaacaggcggaagttgtgttggtggtcggttcgaaaaactcctccaactccaaccgtctggcggagctggcccagcgtatgggcaaacgcgcgtttttgattgacgatgcgaaagatatccaggaagagtgggtgaaagaggttaaatgcgtcggcgtgactgcgggcgcatcggccccggatattctggtgcagaatgtggtggcacgtttgcagcagctgggtggtggtgaagccattccgctggaaggccgtgaagaaaatattgttttcgaagtgccgaaagagctgcgtgtcgatattcgtgaagtcgattaaccatggggtaccgcggccgctaaggaggtataacatatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccaccagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataagcggccgccctaggggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttgatca